(19) United States
(12) Patent Application Publication
(10) Pub. No.: US 2003/0152647 A1
(43) Pub. Date: Aug. 14, 2003

(54) Safe botanical drug for treating malignant pleural effusion and cancer and increasing immune function

(76) Inventor: Yaguang Liu, Flushing, NY (US)

Correspondence Address: Yaguang Liu, 67-08 168th Street, Flushing, NY, 11365, US

(21) Appl. No.: 10/062,316

(22) Filed: Feb. 4, 2002

(51) Int. Cl. A61K 35/78

(52) U.S. Cl. 424/725

(57) ABSTRACT

Three new safe pharmaceutical compositions in accordance with the present invention are for treatment and prevention of malignant pleural effusion and increase immune function comprise Lan Xiang Xi (LX) and polysaccharide of Dang Gui.

Methods of treating and preventing cancer cells include inhibiting oncogenes, increasing activity of tumor suppressor, inducing differentiation of cancer cells, inhibiting cancer cells proliferation, inducing apoptosis of cancer cells, inhibiting growth of transplanted cancer and inhibiting cancer incidence, etc. In general, anticancer drug always decreases immune function. However, LX and LX+PDG can inhibit cancer also increase immune function at the same time. Also, LX and DG containing soybean-liposomes is safer than LX and DG.

### BACKGROUND OF THE INVENTION

This invention relates to new safe botanical drug, which is treatment and prevention of malignant pleural effusion and cancer and increase immune function. Specifically, this invention provides the safe botanical drug comprising Polysaccharide of Dang Gui (PDG) and Lan Xiang Xi (LX). New botanical drug, which treats and prevents cancer through killing cancer cells and increasing immune function at same time, is safe.

### DESCRIPTION OF PRIOR ART

Cancer is the second leading cause of death in the United States, and the incidence of cancer continues to climb annually. In recent years, about 1 million new cases of cancer are diagnosed yearly in the U.S. About half million of people and 7 million people of annual died in the US and in the world, respectively. A lot of anticancer drugs including chemical and antibiotics have effects to kill cancer cells. But it also kills off some normal human cells, appears many kinds of side effects, among them the inhibition of bone marrow and tract are the most common. For example, Cyclophosphamide is a chemotherapeutic drug, which is highly effective against a wide range of human cancers. Unfortunately, it does damage to hemocytogenesis organs, alimentary tract and decreasing immune function. Adriamycin, a sensitive anticancer antibiotic, has seriously cardiotoxicity. Therefore, decreasing toxicity of anticancer drugs is very important.

Many reports indicated that the side effects of plant's anticancer drugs are lower than chemical and antibiotic's anticancer drugs. Therefore, the development of plant drug has progressed very fast now. Taxol, for example, is a novel anticancer plant drug isolated from the needles and bark of the western yew, Taxus brevifolia. It is the prototype for a new class of antitumor drugs, which are characterized by their capacity to promote the assembly of microtubules. Clinical trials conducted in the late 1980s and early 1990s demonstrated impressive clinical activities against advanced ovarian and breast cancer. Taxol still has some side effects, for example, myelosuppression, diarrhea, emesis, oligospermia, cellular depletion in lymphoid tissues, changes in serum hepatic enzymes, and elevations in cholesterol and triglycerides were observed. More important, taxol has two big problems. The first problem is that natural source of taxol is very limited. And the second problem is that taxol is a poor-water soluble. Vehicles for parental administration on taxol cause serious side effects.

Recently a number of gene expression systems have been developed that can be regulated by the administration of specific small molecule drugs. It is important that the small molecule drugs are easily administered. Some of the earliest systems include exposure to heavy metal, and steroid hormones, but they are not suited for in vivo human. These systems, however, are suitable to study for the effect of small molecular drugs on controllable oncogenes, and to study for the treatment of cancer.

So far, no one drug has been succeeded to treat or prevent cancer by control cancer cells and without adverse side effects.

### SUMMARY OF THE INVENTION

This invention relates to new safe botanical drug, which is treatment and prevention of treating malignant pleural effusion, increase immune function and treating cancer. Specifically, this invention provides the safe botanical drug comprising polysaccharide of Dang Gui (PDG) and Lan Xiang Xi (LX).

New botanical drug, which treats and prevents cancer through killing or controlling cancer cells and increasing immune function in same time, is safe.

Methods of killing or controlling cancer cells include inhibiting oncogenes, increasing activity of tumor suppressor, inducing differentiation of cancer cells, inhibiting cancer cells proliferation, inducing apoptosis of cancer cells, inhibiting growth of transplanted cancer and inhibiting cancer incidence.

### DETAILED DESCRIPTION OF THE INVENTION

Recent developments in cancer research, more specifically, in molecular biology, biochemical aspects, indicated that controlling cancer cells is more important than killing cancer cells. For example, control oncogene expression, apoptosis, and differentiation of cancer cells are several key steps in process of control cancer. The control of oncogene expression, apoptosis and differentiation of cancer is focus in cancer biology and molecular pharmacology of anticancer drug. For example, concentration of oncogene c-myc and c-myb RNA is rapid increased in human leukemia and other cancer cell, and a rapid decline in the expression of c-myc and c-myb RNA was seen in cancer cells induced to differentiate. To inhibit oncogene expression, apoptosis and differentiation of cancer cells can control and destroy carcinogenesis and stop cancer progression.

It is more important that LX is successfully used in clinic for malignant pleural effusion and treatment cancer. For example, at least have 600 cases clinic reported that LX treated patients with malignant pleural effusion higher than 70%. Also, LX could effective treat lung cancer and increasing immune function.

The following specific examples will provide detailed illustrations of methods of producing relative drugs, according to the present invention and pharmaceutical dosage units containing relative drugs. Moreover, examples described pharmaceutical characters of drugs, which demonstrated its effectiveness in control of cancer cells. These examples are not intended, however, to limit or restrict the scope of the invention in any way, and should not be construed as providing conditions, parameters, reagents, or starting materials which must be utilized exclusively in order to practice the present invention.

### EXAMPLE 1 EXTRACTION OF POLYSACCHARIDES OF DANG GUI (PDG)

2,000 ml of water was added to 1,000 g of dry powder of Dang Gui, a Chinese herb. The mixture was heated to boil and simmered for one and one-half hours after boiling. This water extraction was repeated once and the two extracts were combined and filtered. The filtrate was concentrated under reduced pressure to approximately 500 ml and 95% ethanol was added to the concentrate to yield a final alcohol concentration of 60%. The resulting solution was filtered to recover a precipitate which was dissolved in an appropriate amount of water, the resulting solution was filtered to remove residue and a filtrate was saved. The filtrate was concentrated under reduced pressure to 200 ml and 95% ethanol added to the concentrate to yield a final alcohol concentration of 80%. The solution was then discarded and the precipitate was washed three times with 95% ethanol and then twice with acetone and ether consecutively. The product was vacuum dried, and the resulting powder was polysaccharides of Dang Gui.

### EXAMPLE 2 LX EXTRACTION

One kg of plant powder was extracted 5 L of water at room temperature for 12 hours. The powder of plant named Dryobalanops aromatica Gaerin or Wen E Shu was recovered by filtration. Filtrate A was saved and the powder filtercake was extracted with 4 L of water at room temperature for 10 hours. The mixture was filtered. Filtrate B was saved and powder filtercake was extracted for 3 L of water at room temperature for 8hours. The mixture was filtered and filtrate C was saved. Filtrate A, B, and C was combined at distilled under reduced pressure for 32 hours. The distilled mixture was separated. The oil fraction was saved and kept temperature at 0° C. The oil distilled under reduced pressure, (50°-80° C./40 Pa) and fraction A was collected. Fraction A distilled under reduced pressure (76°-78° C./40 Pa) and fraction B was collected. Fraction B was Elemne and Fraction B was then chromatographed on silica gel G, using petroleum ether as developing solvent. The solvent collected and dried. The final product is LX.

LX has the following chemistry data. Molecular form: C₁₅H₂₄ Molecular weight. 204 Mp: 114˜118° C. [α]¹⁶: −15° IRν^{KBr} cm⁻¹: 3090, 2975, 2860, 1642, 1440, 1375, 1002, 910, 888 (C═CH₂); PMR (CCl₄) δ: 0.97 (3H, s), 1.7 (6H, s), 4.4˜5.6 (6H, m), 5.7 (1H, dd, ═OH₂); MSm/e (%): 204 (M⁺), 147 (33), 121 (41), 107 (54), 93 (89), 81 (100), 79 (44), 68 (74), 67(52), 55 (41), 53 (33), 41(52).

### EXAMPLE 3 INJECTION SOLUTION OF LX

Four (4) volumes of 95% ethanol were added to LX. The solution was allowed to stand for 24 hours and then filtered. The filtrate distilled under reduced pressure and the ethanol recovered. Six volumes of 95% ethanol were added to the residue. After standing for another 24 hours, the solution was filtered. The filtrate distilled under reduced pressure and ethanol recovered. The residue was then distilled until there was no remaining smell of alcohol. Sufficient distilled water was added to dissolve the residue, and the solution was filtered to remove any undissolved material. Pharmaceutical glycerin was added to the solution. The solution was then fine filtered, and the volume adjusted to 500 liters with distilled water. After additional fine filtering, the solution was sealed in 2 ml sterile ampoules, which were further sterilized and sealed. Each ampoule contained 5 mg of LX per milliliter of solution.

### EXAMPLE 4 PREPARATION OF LX-CONTAINING STERICALLY STABILIZED LIPOSOMES (LX-SSL)

Hydrogenated phosphatidyl choline (PC), phosphatidyl glycerol (PG), and phosphatidyl serine (PS) were extracted from soybean. All above lipids were finally purified on silicic acid columns, shown to be pure by thin-layer chromatography and stored in chloroform in sealed ampules under nitrogen until use. Phospholipids mixed with cholesterol (CHOL) and long-chain alcohol. The solvent was removed under reduced pressure by a rotary evaporator. The lipids were then purged with nitrogen. Lipids were redissolved in the organic phase and reversed phase will be formed. LX-containing solution was added at these lipid systems, and resulting two-phase system was sonic 3 minutes until the mixture homogeneous that did not separate for at least two hours. A typical preparation contained 3.3×10⁻³ M of phospholipid and 3.3×10⁻³ M of cholesterol in 1 litre of phosphate-buffered saline and 3 liters of solvent. LX-SSL were sealed and sterilized. The size of the vesicles was determined by a dynamic light cattering technique. When PG/PC/CHOL were 1:4:5, diameter of liposomes was 20-50 nM (range). LX-SSL was very stabilized in at least nine months.

So far, many articles reported drug-containing liposomes. However, liposomes are not stabilized enough. Therefore it is difficult to be used for pharmaceutical industry. In accordance with this invention, LX-SSL is very stabilized in at least nine months. Therefore LX-SSL can be used in industry. LX-SSL can enhance targeting and improve pharmaceutical activity of LX. The results show that LX-SSL is safer than LX. LX+PDG-SSL is safer than LX+PDG. Also it is very important that all lipids are extracted from soybean. Therefore it is very safe for human being. In fact, many articles reported lipids, which used for drug-containing liposomes, are syntheses by organic chemistry. However synthetic lipids have some side effects, therefore, according to this invention, methods of preparing of LX-SSL and phospholipids, which extracted from soybean, are very safe.

### EXAMPLE 5 PREPARATION OF BOTANICAL DRUG

1 kg polysaccharide of Dang Gui and 1 kg of LX produced in accordance with the preceding example, were thoroughly mixed and agitated until a homogeneous mixture of the two components was obtained. Dosage form was capsule or tablet, which includes in addition pharmaceutically acceptable binders and excipients.

### EXAMPLE 6 PREPARATION OF PDG-CONTAINING STERICALLY STABILIZED LIPOSOMES (PDG-SSL)

Hydrogenated phosphatidyl choline (PC), phosphatidyl glycerol (PG), and phosphatidyl serine (PS) were extracted from soybean. All above lipids were finally purified on silicic acid columns, shown to be pure by thin-layer chromatography and stored in chloroform in sealed ampoules under nitrogen until use. Phospholipids mixed with cholesterol (CHOL) and long-chain alcohol. The solvent was removed under reduced pressure by a rotary evaporator. The lipids were then purged with nitrogen. Lipids were redissolved in the organic phase and reversed phase will be formed. PDG-containing solution was added at these lipid systems, and resulting two-phase system was sonicated 3 minutes until the mixture homogeneous that did not separate for at least two hours after sonicated. A typical preparation contained 3.3×10⁻³ M of phospholipid and 3.3×10^{31 3} M of cholesterol in 1 litre of phosphate-buffered saline and 3 liters of solvent. PDG-SSL were sealed and sterilized. The size of the vesicles was determined by a dynamic light cattering technique. When PG/PC/CHOL were 1:4:5, diameter of liposomes was 20-50 nM (range). PDG-SSL was very stabilized in at least nine months.

So far, many articles reported drug-containing liposomes. However, liposomes are not stabilized enough. Therefore it is difficult to be used for pharmaceutical industry. In accordance with this invention, PDG-SSL is very stabilized in at least nine months. Therefore PDG-SSL can be used in industry. PDG-SSL can enhance targeting and improve pharmaceutical activity of PDG. In fact, PDG-SSL is safer than PDG. Also it is very important that all lipids are extracted from soybean. Therefore it is very safe for human being. In fact, many articles reported lipids, which used for drug-containing liposomes, are syntheses by organic chemistry. However synthetic lipids have some side effects, therefore, according to this invention, methods of preparing of PDG-SSL and phospholipids, which extracted from soybean, are very safe.

### EXAMPLE 7 THE EFFECT OF LX AND PDG ON CONTROL OF ONCOGENES

Human myeloblastic leukemic cell (ML-1) had been described previously (8). Cells were maintained in suspension culture in RPMI 1640 medium supplemented with 7.5% heat-inactivated FBS. Cells growth and viability were assayed by hemocytometer using trypan-blue dye exclusion. RNA was isolated by the CsCl gradient modification. RNA pellets were washed twice by reprecipitation in ethanol and quantitated by absorbency at 260 nM. RNA analyzed by electrophoresis of 15 μg of RNA through 1.2% agarose formaldehyde gels followed by northern blot transfer to nitrocellulose.

Single-standard uniformly labeled DNA probes were prepared. Probe of c-myc was a 1.7 Kb cla-Eco RI restriction fragment containing the 3′exon region of human c-myc and probe of c-myb was 1.0 Kb myb-specific Bam HI fragment. Probes for n-ras contained DNA fragments using a modification of the PCR technique. Probes for myb, myc and n-ras were isolated by electrolution. The isolated fragments were labeled to high specific activity with [α³²P]-dCTP (3000 ci/mmol). Prehybridization of the filter was performed. The hybridization mixer contained 50,000 cpm of probe. The probes were hybridized at 58° C. in 15 mM NaCl, 1.5 nM sodium citrate for 3 hours. After hybridization, they were exposed to XAR-5 film. Oncogene expression was quantitated by densitometer scanning of the autoradiography.

The effect of LX on oncogene was determined. The results are summarized in the tables as below.

TABLE 1
The effect of LX and PDG concentration on inhibition of oncogenes

| LX | | | |
|---|---|---|---|
| concentration | Inhibition (%) | | |
| (ng/ml) | c-myb RNA | c-myc RNA | RAS |
| Saline | 0 | 0 | 0 |
| LX (10) | 25.0 ± 2.8* | 24.2 ± 2.8* | 15.2 ± 2.0* |
| LX (50) | 68.5 ± 7.8** | 78.0 ± 8.5** | 28.2 ± 3.8** |
| LX (50) + | 92.2 ± 12.50*** | 90.2 ± 10.5** | 45.8 ± 6.2 |
| PDG (50) | | | |

nM = 10⁻⁹ M;
*P < 0.05,
**P < 0.01,
***P < 0.001 compared with LX

This study clearly indicated that LX and LX+PDG could significantly inhibit oncogenes of cancer cells. Cancers would be suitable targeted for gene-directed therapy and the present study has been directed toward the suppression of oncogene activity in cancers. Cellular oncogenes encode proteins have important function in differentiation of cancer cells. The principal functions of c-myc are the induction of proliferation and the inhibition of terminal differentiation in many cells. Over-expression of myc commonly occurs in a wide range of tumors.

### EXAMPLE 8 THE EFFECT OF LX AND PDG ON DECREASING OF TYROSINE KINASE

In general, very low levels of TK are expressed in normal cells and high levels of TK are expressed in cancer cells. Many evidences have been accumulated that the dysfunction of cellular oncogenes is a cause of human cancers. Therefore, a drug, which inhibits the activity of TK, can provide a new way to overcome cancer.

Methods

Cells. L1210 and P388 cells were grown at 37° C. on medium RPMI-1640 without antibiotics and supplemented with 10% horse serum. Cultures were diluted daily to 1×10⁵ cells/ml with fresh growth medium. From a culture initiated with cells from ascitic fluid obtained from a mouse 5 days after implantation with in vivo-passage leukemia, a stock of ampoule containing 10⁷ cells/ml in growth medium plus 10% dimethyl sulfoxide was frozen and stored in liquid nitrogen. Cultures were started from the frozen stock and were passage for no more than 1 month. L1210 and P388 cells were grown at 37° C. on medium RPMI-1640 supplemented with 10% calf serum, 10,000 unit/ml of Penicillin and 10,000 unit/ml of Streptomycin. 1×10⁶/ml cells were placed in culture with different concentrations of LX. Then the cell suspension was incubated at 37° C. in a humidified atmosphere of 5% CO₂-95% air for the indicated time. Reactions were terminated by addition of 3 ml of cold Earle's buffer. Cells were lysed, precipitated with 10% trichloroacetic acid (TCA) and filtered onto glass fiber filters. The filters were washed with phosphate-buffered saline and placed in scintillation vials, and radioactive emissions were counted.

H-60 leukemia cells were plated at a density of 5×10⁵ cells in 60-nm dished, and divided control and treatments groups for incubation 24 hours at 37° C. with 5% CO₂. The cells were collected and washed twice with phosphate-buffered saline, and resuspended at density of 10⁶ cells/ml in 5 mM HEPEs buffer (pH 7.4). The cells were then resuspended in 1 ml of buffer containing 5 mM HEPES (pH 7.6), 1 mM MgCl₂ and 1 mM EDTA, then placed on ice bath. The cell membrane was disrupted by ultra sound and centrifuged at 1000×g for 10 minutes. The supernatant was ultra centrifuged at 30,000×g for 30 minutes at 4° C. The pellet was resuspended in 0.3 ml of buffer containing 25 mM HEOES, centrifuged at 12,000×g for 5 minutes. The resulting supernatant was used for TK assay. Content of protein was determined. 10 μg of protein placed in 20 mM HEPES (pH 7.6), 15 mM MgCl₂, 10 mM ZnCl2 and 5% (v/v) Nonidet P-40. After 5 minutes incubation at 25° C., the reaction was initiated by the addition of 25 μM [γ³²P] ATP (3 ci/mmol). After 10 minutes, the reaction was stopped by the addition of 20 mM cold ATP. 50 μl of the mixtures were spotted on glass microfiber filter discs and washed three times with cold trichloroacetic acid (TCA), contained 10 mM sodium pyrophosphate. Air dried. Radioactivity was determined by liquid scintillation spectrometry. The net TK activity was determined after correcting for endogenous TK activity.

The present study clearly demonstrated that LX and LX+PDG significantly reduced in TK activity.

TABLE 2
Effect of LX and PDG on TK activity of HL-60 leukemia cells

| Group | % of control activity |
|---|---|
| Control | 100 |
| LX (10 ng/ml) | 8.0 |
| LX (50 ng/ml) | 32.5 |
| LX (50 ng/ml) + | 94.2 |
| PDG (50 ng/ml) | |

Value represents the mean of two experiments each done in duplicate; the range was less than 5% of the mean.

### EXAMPLE 9 LX AND PDG CONTROLS DIFFERENTIATION OF CANCER CELLS

Human promyelocytic leukemia cells (HL-60) were grown in RPMI Medium 1640 supplemented with 10% (v/v) heat-inactivated FBS (56° C. for 30 min) at 37° C. in a humidified 95% air/5% CO₂ atmosphere. Cells were seeded at a level of 2×10⁵ cells/ml. Cells were allowed to attain a maximum density of 1.2×10⁶ cells/ml before being passed by dilution into fresh medium to a concentration of 2×10⁵ cells/ml.

Differentiation was induced in HL-60 cells by treatment with 0.1 μg/ml (10×10⁻⁷ g/ml) of LX or PDG. Cell differentiation was measured by the ability of cells to reduce NBT.

TABLE 3
Induced differentiation of LX and PDG on cancer cells

| Treatment | NBT %* |
|---|---|
| None | 0 |
| LX | 68.5 ± 7.2 |
| LX + PDG | 72.8 ± 8.5 |

*NBT represents the percentage of cells capable of reducing NBT.

The results of Table 4 indicate that LX and LX+PDG markedly induced differentiation of human cancer cells. Increasing NBT % means that cancer cells are differentiation.

### EXAMPLE 10 EFFECTS OF LX AND PDG ON TUMOR CELLS PROLIFERATION

Human tumor cell lines: Hela leukemia HL-60, malignant melanocarcinoma B 16, oral epidermoid carcinoma (KB), lung carcinoma (A549), breast carcinoma MCF-7, adenocarcinoma of stomach.

Animal tumor cell lines: Walker carcinoma, LLC-WRC-256, malignant melanoma (RMMI 1846), 3T3, and S-180 sarcoma (CCRF-180). All lines were routinely cultured in the RPMI1640 medium supplemented 20% fetal calf serum. The experiment was carried out in 96 microplate, each well had 5×10⁵ cells and given desired concentration of 1 μg/ml (1×10⁻⁶ g/ml) drug. Then the plate was incubated at 37° C. in an atmosphere of humidified air enriched with 5 percent carbon dioxide for 72 hours. Concentration of LX or PDG is 50 ng/ml.

Inhibition percent rate of tumor cell proliferation was obtained according to the bellow formula.

$$Inhibition percent rate = \frac{Control \text{-} Test }{Control} \times 100 \%$$

LX inhibited tumor cells growth significantly. Percent rates of inhibition were all more than 70% in all cancer cells by LX.

TABLE 4
Effect of LX and PDG on inhibiting growth cancer cells

Inhibition (%)

| Cell line | LX | LX + PDG |
|---|---|---|
| Control | — | — |
| Human cells | | |
| HL-60 | 78.5 ± 8.2 | 82.8 ± 9.0 |
| Hela | 70.7 ± 8.0 | 84.0 ± 9.8 |
| KB | 73.8 ± 8.9 | 80.8 ± 7.8 |

### EXAMPLE 11 EFFECT OF LX AND PDG ON APOPTOSIS OF CANCER CELLS

Human promyelocytic leukemia cells (HL-60) were grown in RPMI Medium 1640 supplemented with 10% (v/v) heat-inactivated FBS (56° C. for 30 min) at 37° C. in a humidified 95% air/5% CO₂ atmosphere. Cells were seeded at a level of 2×10⁵ cells/ml. Cells were allowed to attain a maximum density of 1.2×10⁶ cells/ml before being passed by dilution into fresh medium to a concentration of 2×10⁵ cells/ml.

Apoptosis determined by the following process: Cell pellets containing 5×10⁶ cells ere fixed with 2.5% glutaraldehyde in cacodylate buffer (pH 7.4), dehydrated through graded alcohol, and infiltrated with LX-112 epoxy resin. After overnight polymerization at 60 C 1-μm sections were cut with glass knives using a LKB Nova microtome. The sections were stained with 1% toluidine blue and coverslipped. In addition, experimental examples were stained with May-Grunwald-giemsa stain for the demonstration of apoptosis.

DNA electrophoresis: Untreated and treated HL-60 cells collected by centrifugation, washed in phosphate buffered saline and resuspended at a concentration of 5×10⁶ cells and 0.1% RNase A. The mixture was incubated at 37° C. for 30 min and then incubated for an additional 30 min at 37° C with 1 ml protease K. Buffer was added and 25 μl of the tube content transferred to the Horizontal 1.5% agarose gel electrophoresis was performed at 2 V/cm. DNA in gels visualized under UV light after staining with ethidium Bromide (5 μg/ml).

DNA fragmentation assays: DNA cleavage was performed, quantitation of fractional solubilized DNA by diphenylamine assay and the percentage of cells harboring fragmented DNA determined by in labeling techniques. For the diphenylamine assay, 5×10⁶ cells were lysed in 0.5 mL lysis buffer (5 mmol/L Tris-HCl, 20 mmol/L DTA, and 0 5% Triton X-100, pH 8.0) at 4° C. Lysates were centrifuged (15,000 g) for separation of high molecular weight DNA (pellet) and DNA cleavage products (supernatant). DNA was precipitated with 0.5N perchloric acid and quantitated using diphenylamine reagent.

The cell cycle distribution was determined 4 hours after addition of drug and represents mean±SD of 5 independent experiments.

TABLE 5
Effect of LX on apoptosis of cancer cells

| Drug | |
|---|---|
| Concentration (μg) | Apoptosis (%) |
| Control | 0 |
| LX (50) | 70.8 ± 8.2 |
| LX (50) + PDG (50) | 80.8 ± 8.5 |

Data of table 5 indicated that LX and LX+PDG could significantly induce apoptosis.

### EXAMPLE 11 THE EFFECT OF LX AND PDG ON THE GROWTH OF ANIMAL TRANSPLANTED TUMOR

Experimental Procedure:

Male mice, weight 20-22 g, were used in the experiment. 1×10⁷tumor cells were injected to mouse and LX injected intraperitoneally began second day. All mice were sacrificed on the 12th days, isolated the tumor and weighed and calculated the inhibition rate of tumor weight.

Results:

The effect of LX on the growth of animal transplanted tumor as illustrated by the table 6. LX 20 mg/kg could inhibit the growth of S180, ECS, HCS, ARS, U-14 and L615 transplanted tumor.

TABLE 6
Inhibition rate (%) of transplanted tumor

| | Inhibition (%) | |
|---|---|---|
| Transplanted tumor | LX (50 ng/ml) | LX (50 ng/ml) + PDG (50 ng/ml) |
| Control | — | — |
| L1210 | 74 ± 9.0 | 87.0 ± 9.8 |
| Lewis | 72 ± 8.8 | 89.0 ± 10.1 |
| S180 | 70 ± 9.0 | 83.0 ± 9.8 |
| Walker 256 | 75 ± 8.0 | 92.5 ± 10.8 |

Data of table 6 indicated that LX and LX+PDG could significantly inhibit growth of tumor.

### EXAMPLE 12 LX AND PDG INHIBITED TUMOR INCIDENCE IN VIVO

The capacity of tobacco-specific nitrosamine 4-(methylnitrosamino)-1-(3-pyridyl)-1-butanone (NNK) to induce tumor incidence was recognized.

Methods:

Every group had 20 mice. For treatment group, each mouse was gave LX and PDG by injection at dose of 20 mg/kg daily For control group, each mouse was gave same volume of physiological saline. Three days later, mice were gave 10 μmol NNK (in 0.1 ml saline) by i.p. injection. Sixteen weeks after these treatments the mice were killed and pulmonary adenomas were counted. The statistical significance of bioassay data was determined by student's test.

TABLE 7
Effects of LX and PDS on NNK-induced lung tumorigenesis

| Group | Tumor incidence (%) | P |
|---|---|---|
| Control | 100 | — |
| LX | 28.2 ± 5.0 | <0.01 |
| LX + PDG | 20.0 ± 3.0 | <0.01 |

Data of table 7 indicated that LX and LX+PDG have a significant tumor incidence.

### Example 13 THE EFFECT OF LX AND PDG ON PHAGOCYTOSIS OF PERITONEAL MACROPHAGE IN MICE

Added 0.02 ml of 5% washed chick red blood cell suspension to 0.5 ml of the peritoneal exudate, shaken gently to mix and incubated at 37° C. for 5 minutes. Dipped two coverslips, closed to each other, in the above mixture and incubate for 30 minutes for the migration of the macrophages along the coverslips then fixed and stained with Sharma stain. Examined microscopically for: Phagocytic rate—number of macrophages with phagocytized chick red blood cells per 100 macrophages counted.

Method of animal model was regular. The drugs were injected subcutaneously daily for 7 days.

TABLE 8
Effect of LX and PDG on immune function

| Group | Number of samples | Phagocytic rate (x + SD, %) |
|---|---|---|
| Control | 10 | 34.10 ± 1.45 |
| CY | 10 | 7.52 ± 0.46 |
| CY + LX | 10 | 25.6 ± 3.20* |
| CY + LX + PDG | 10 | 30.8 ± 5.60* |

*P < 0.01 compared with treatment group

The counting result showed that LX could increase the phagocytic rate. It suggested that LX and PDG could improve the nonspecial immune function in mice. And LX+PDG has more highly increasing immune function.

### EXAMPLE 14 THE EFFECT OF LX AND PDG ON THE SUBPOPULATION OF LYMPHOCYTE

As same as in Example 13, injected CY as the immune inhibitor for each mouse with one time daily and 7 times totally. The analysis of the subpopulation of T lymphocyte was carried out using fluorescein labeled monoclonal antibody of mouse CD₈ and CD₄ and determined cells by flow cytometer. The results are listed in Table 9.

TABLE 9
The effect of LX and PDG on the subpopulation of T lymphocyte

| Group | Cases | CD₈ | CD₄ |
|---|---|---|---|
| Control | 10 | 25.4 | 38.7 |
| LX | 10 | 19.0 | 20.2 |
| LX + PA | 10 | 33.0 | 36.8 |

Table 9 indicated that LX and LX+PDG could strongly increase immune function.

### EXAMPLE 15 SAFETY OF COMPOSITION

1. The acute LD₅₀ of LX was found to be 340 mg/kg injection in abdominal cavity in mice. And LD₅₀ of LX-SSL was 721 mg/kg. LD₅₀ of polysaccharide of Dang Gui for injection with abdominal in mice was higher than 1 g/kg. They are very safe.

2. Each dose for an adult was 50-500 mg. Using 50 kg as the average weight of an adult the dosage of LX, LX-SSL, PDG and PDG-SSL were 1-10 mg/kg, and they are very safe.

3. As to subacute toxicity tests, a dosage corresponding to 50 times the clinical dose of LX was administered continually for two months, and no side effects had been observed. The electrocardiograms and functions of liver and the kidney had not been effect and no injuries whatever had been observed in the tissue slices of the heart, liver, spleen, lungs, kidneys and adrenal.

### EXAMPLE 16 ASSAY AND IDENTIFICATION OF BOTANICAL DRUG

In the present study, we examined LX by chromatography with cambowax column. Temperature was 135° C. Methy salicylate was used as internal standard. The ration of peak arrears was linear function with concentration when between 0.5 to 1.5 mg/ml. Data of LX are listed as below.

y=1.32 χ−0.03, γ=0.9999. Molecular form: C₁₅H₂₄ Molecular weight: 204 Mp: 114˜118° C. [α]¹⁶: −15° IRν^{KBr} cm⁻¹: 3090, 2975, 2860, 1642, 1440, 1375, 1002, 910, 888 (C═CH₂); PMR (CCl₄) δ: 0.97 (3H, s), 1.7 (6H, s), 4.4˜5.6 (6H, m), 5.7 (1H, dd, ═OH₂); MSm/e (%): 204 (M⁺), 147 (33), 121 (41), 107 (54), 93 (89), 81(100), 79 (44), 68 (74), 67 (52), 55 (41), 53 (33), 41(52).

### EXAMPLE 17 PHARMACOKINETICS OF LX

Distribution of LX (100 mg/kg) after injection to rate was determined. The results are listed below table.

TABLE 10
Pharmacokinetics and disposition of LX

| | Concentration/μg/g | | |
|---|---|---|---|
| Organ | 15 min | 30 min | 2 h |
| Heart | 250 ± 32 | 15.8 ± 2.0 | 7.2 ± 1.0 |
| Liver | 125.5 ± 25 | 7.0 ± 0.8 | 0.7 ± 0.08 |
| Spleen | 150 ± 21 | 28 ± 3 | 2.0 ± 0.3 |
| Lung | 99 ± 10 | 10 ± 2.1 | 3.5 ± 0.4 |
| Kidney | 205 ± 31 | 50 ± 6.0 | 12 ± 2.0 |
| Muscle | 50 ± 6.1 | 9.0 ± 1.2 | 3.5 ± 0.4 |
| Stomach | 68 ± 7.0 | 8.9 ± 0.9 | 1.8 ± 0.2 |
| Plasma | 41 ± 5.0 | 6.5 ± 0.8 | 1.0 ± 0.2 |

The above data indicated that absorption and elimination of LX was rapid. LX, therefore, is a safe botanical drug.

The preparation of drugs, which can be accomplished by the extraction methods, set forth above or any conventional methods for extracting the active principles from the plants. The novelty of the present invention resides in the mixture of the active principles in the specified proportions to produce drugs, and in the preparation of dosage units in pharmaceutically acceptable dosage form. The term “pharmaceutically acceptable dosage form” as used hereinabove includes any suitable vehicle for the administration of medications known in the pharmaceutical art, including, by way of example, capsules, tablets, syrups, elixirs, and solutions for parenteral injection with specified ranges of drugs concentration.

In addition, the present invention provides novel methods for treating and preventing a variety of cancer conditions and control cancer cells with produced safe pharmaceutical agent. It will thus be shown that there are provided compositions and methods which achieve the various objects of the invention and which are well adapted to meet the conditions of practical use.

As various possible embodiments might be made of the above invention, and as various changes might be made in the embodiments set forth above, it is to be understood that all matters herein described are to be interpreted as illustrative and not in a limiting sense.

What is claimed as new and desired to be protected by letter patent is set forth in the appended claims:

1. A safe botanical drug for treatment and prevention of malignant pleural effusion and cancer, and enhancement immune function comprises polysaccharide of Dang Gui (PDG) and Lan Xiang Xi (LX).

2. A safe botanical drug of claim 1 wherein the amount sufficient to inhibit oncogenes, induce differentiation of cancer cells, inhibit cancer cells proliferation, induce apoptosis of cancer cells, inhibit growth of transplanted tumors, and inhibit cancer incidence, is about 50-200 mg of LX derivate.

3. A safe botanical drug of claim 1 wherein the amount sufficient to enhance immune function and inhibit oncogenes and cancer incidence, is about 25-100 mg of PDG.

4. A safe botanical drug of claim 1 wherein the amount sufficient to inhibit oncogenes, induce differentiation of cancer cells, inhibit cancer cells proliferation, induce apoptosis of cancer cells, inhibit growth of transplanted tumors, and inhibit cancer incidence, is about 50-200 mg of LX and PDG derivate.

5. A composition, according to claim 1, wherein said LX is extracted from plant of Dryobalanops aromatica Gaerin or Wen E Shu.

6. A composition, according to claim 1, wherein said polysaccharides of Dang Gui (PDG) is extracted from Dang Gui.

7. A pharmaceutical composition of claim 1, wherein said dosage form is comprises LX and a pharmaceutical acceptable carrier.

8. A pharmaceutical composition of claim 1, wherein said dosage form is comprises LX and PDG and a pharmaceutical acceptable carrier.

9. A composition in accordance with claim 7 in injection solution dosage unit form.

10. A composition in accordance with claim 8 in injection solution dosage unit form.

11. A process for producing LX, which used for treatment and prevention of malignant pleural effusion and cancer and enhancement immune function of claim 1 is comprising:
a. extracting a powder of Drybalanops aromatica Gaerin or Wen E Shu with water;
b. the filtrate was saved and filtercake extracted with water again,
c. the filtrate combined and distilled under pressure;
d. the distilled mixture was separated and oil fraction was kept at 0° C.;
e. the oil distilled under reduced pressure (50°-80° C./40 Pa) and fraction was collected;
f. the fraction was distilled under reduced pressure (76°-78° C./40 Pa) and fraction B was collected;
g. the fraction B was chromatographed on silica gel G and using petroleum ether as developing solvent;
h. the solvent collected and dried; and
i. the final product is LX.

12. A process for producing polysaccharides of Dang Gui (PDG), which used for control cancer cells of claim 1 is comprising:
a. extracting a powder of Dang Gui with water by boiling first and then simmering for 1.5 hours;
b. filtering the extract from the powder residue;
c. concentrating the extract under reduced pressure;
d. adding 95% ethanol to the concentrate of (c), to produce a 60% ethanol solution to form a precipitate;
e. separating the precipitate from the ethanol solution;
f. dissolving the precipitate in water,
g. concentrating the filtrate of (f) under reduced pressure;
h. adding 95% ethanol to the concentrated filtrate to yield an 80% ethanol solution;
i. cooing the 80% ethanol solution to form a precipitate;
j. separating the precipitate and drying same under vacuum to yield a final powder;
k. washing said product with 95% ethanol three times;
l. washing the product of (k), consecutively with acetone and ether twice;
m. vacuum drying the product of (l) and.
n. The final product is PDG.

13. A safe botanical drug, according to claim 1, wherein said LX determined by HPLC and the special index is y=1.32 χ−0.03, γ=0.9999.

14. A safe botanical drug comprising a polysaccharide of Dang Gui containing soybean-liposomes (DGL) and LX-containing soybean-liposomes (LXL) is used for treatment and prevention of malignant pleural effusion and cancer, and enhancement immune function.

15. The safe botanical drug of claim 14, wherein the amount sufficient for treatment and prevention of malignant pleural effusion and cancer, and enhancement immune function is about 20-40 mg of DGL and 20-40 mg of LXL.

16. A safe botanical drug in accordance with claim 14 wherein said liposomes contained hydrogenated phosphatidyl choline (PC), phosphatidyl glycerol (PG), and phosphatidyl serine (PS), which purified from soybean.

17. A process for producing DGL and LXL, which used for treatment and prevention of malignant pleural effusion and cancer, and enhancement immune function of claim 13 comprising:
a. Phosphatidyl choline (PC), phosphatidyl glycerol (PG), and phosphatidyl serine (PS), were purified from soybean;
b. PC, PG, and PS were purified on silicic acid columns;
c. Pc, PG, and PS mixed with cholesterol (CHOL) and long-chain alcohol;
d. Lipids were dissolved in the organic phase and reversed phase would be formed;
e. PDG or LX was added at lipid systems and resulting two-phase system was sonicated 3 minutes; and
f. PDGL or LXL was sealed and sterilized.

18. A safe composition to claim 14 wherein said DGL used for enhancement of immune function and inhibiting oncogenes and cancer incidence.

19. A safe composition to claim 14 wherein said LXL used for inhibiting oncogenes.

20. A safe composition to claim 14 wherein said LXL used for inducing differentiation of cancer cells.

21. A safe composition to claim 14 wherein said LXL used for inhibiting cancer cells proliferation.

22. A safe composition to claim 14 wherein said LXL used for inducing apoptosis of cancer cells.

23. A safe composition to claim 14 wherein said LXL used for inhibiting growth of transplanted tumors.

24. A safe botanical composition to claim 14 wherein said LXL used for inhibiting cancer incidence.

25. A safe botanical composition to claim 14 wherein said PDGL enhancement of immune function, inhibiting oncogenes and cancer incidence.

26. Safe botanical drug (LX, LXL and PDG), which used for treatment and prevention of malignant pleural effusion and cancer, and enhancement immune function, comprises the following toxicological and pharmacokinetics indexes:
a. LD₅₀ of LX is 340 mg/kg.
b. LD₅₀ of LXL is 721 mg/kg.
c. LD₅₀ of PDG is >1 g/kg, and
d. Elimination of LX is rapid.
